(19) United States
(12) Patent Application Publication
(10) Pub. No.: US 2011/0223666 A1
(43) Pub. Date: Sep. 15, 2011

(54) CYTOPLASM EXPOSURE ADDITIVE AND METHOD FOR EXPOSING PARTICLES DELIVERED INTO CELL TO CYTOPLASM FROM ENDOCYTIC VESICLES IN INTACT CELL

(75) Inventor: Dae Joong Kim, Daejeon (KR)

(73) Assignee: MEDISCOV INC., Daejeon (KR)

(21) Appl. No.: 13/128,327

(22) PCT Filed: Nov. 9, 2009
(86) PCT No.: PCT/KR09/06539

§ 371 Date: May 9, 2011

(30) Foreign Application Priority Data
Nov. 10, 2008 (KR) 10-2008-0111308

(51) Int. Cl. C12N 5/071; C07C 39/15; C07D 207/448; C07C 47/04; C07C 43/303; C07C 31/04; C07C 31/08; C01C 1/16; B82Y 5/00

(52) U.S. Cl. 435/375; 568/729; 548/548; 568/448; 568/599; 568/840; 423/470; 977/773; 977/904

(57) ABSTRACT

The present invention relates to a method of exposing particles to cytoplasm comprising introducing particles into a live cell; allowing the live cell to contact a cytoplasm exposure additive which can expose the particles from endocytic vesicles to cytoplasm in the cell with maintaining its physiological, biochemical, or biological environment as undamaged; and allowing the particles to be exposed from the endocytic vesicles to the cytoplasm. The present invention is advantageous in that particles delivered into cells can be effectively exposed to cytoplasm from endocytic vesicles in intact cells which maintain their physiological, biochemical, or biological environment as undamaged.

### TECHNICAL FIELD

The present invention relates to a cytoplasm exposure additive and a method for exposing particles delivered into a cell to cytoplasm from endocytic vesicles, more particularly to a cytoplasm exposure additive and a method for exposing particles delivered into a cell to cytoplasm from endocytic vesicles effectively in an intact cell where physiological, biochemical, or biological environment is maintained as undamaged.

### BACKGROUND ART

Cell membrane or plasma membrane consists of semi-permeable lipid bilayer and plays a role as a physical barrier between intracellular components and extracellular environments (Albert et al., 2002, Molecular Biology of the Cell. 4th ed., Garland Science). Cell membrane has a characteristic of selective permeability and regulates what enters into and exits from the cell (see http://en.wikipedia.org/wiki/Cell_membrane; downloaded on Oct. 10, 2007). Small molecules or lipid-soluble materials, in other words, hydrophobic and nonpolar materials rapidly pass and diffuse across the lipid bilayer but charged molecules such as ions hardly pass through the cell membrane (Albert et al., 2002, Molecular Biology of the Cell. 4th ed., Garland Science).

Therefore, a technology for delivering materials that are difficult to be delivered into a cell, is important in academic and medical aspects. In other words, a technology for delivering particular materials into a cell is valuable in observing their role in a cell and researches about a cell itself.

Particles such as gold particles and beads have been recently noted in the field of drug development and disease treatment. They have been widely used for labeling cells, a vehicle for delivering materials into cells, and MRI imaging. However, it is not easy to deliver them into cells because their size ranges from several nm to several hundred nm (Berry & Curtis, 2003, Functionalization of magnetic nanoparticles for application in biomedicine. J. Phys. D: Appl. Phys. 36, R198-R206).

To introduce the particles into cells, several methods such as electroporation, microinjection, lipofection using lipid, and intracellular delivery using protein transduction domains, etc. have been tried (Derfus et al., 2004, Intracellular delivery of quantum dots for live cell labeling and organelle tracking Adv. Mater. 16, 961-966; Matuszewski et al., 2005, Cell tagging with clinically approved iron oxides: feasibility and effect of lipofection, particle size, and surface coating on labeling efficiency. Radiology 235, 155-161; Berry & Curtis, 2003, Functionalization of magnetic nanoparticles for application in biomedicine. J. Phys. D: Appl. Phys. 36, R198-R206).

The electroporation has an advantage that it can deliver particles directly to cytoplasm by forming a small hole of nm size on cell membrane by high-voltage electrical pulse. However, there are problems that its efficiency of delivering particles into cells is severely low and the delivered particles are likely to be extremely aggregated (Derfus et al., 2004, Intracellular delivery of quantum dots for live cell labeling and organelle tracking Adv. Mater. 16, 961-966; Rosen et al., 2007, Finding fluorescent needles in the cardiac haystack: tracking human mesenchymal stem cells labeled with quantum dots for quantitative in vivo three-dimensional fluorescence analysis. Stem Cells. 25, 2128-2138).

The microinjection is a technology for introducing particles directly into a single live cell using a micro-needle under a microscope. However, there are the following limitations: a special equipment is required to perform the microinjection; it is very difficult to manipulate; and since particles should be introduced into each cell, it is not efficient to conduct for a plurality of cells.

To overcome these challenges, methods for delivering particles into cells using lipid or protein transduction domains have been recently noted. When lipid or protein transduction domains are used, however, most of particles are introduced into a cell by endocytosis and exist in endocytic vesicles. Hence, the introduced particles in a cell can not be exposed to cytoplasm (Derfus et al., 2004, Intracellular delivery of quantum dots for live cell labeling and organelle tracking Adv. Mater. 16, 961-966; Patel et al., 2007, Cell penetrating peptides: intracellular pathways and pharmaceutical perspectives. Pharm. Res. 24, 1977-1992).

In this regard, when a cargo is delivered into a cell using lipid or protein transduction domains, some methods for exposing the introduced cargo to cytoplasm have been tried and reported. But these methods are directed to delivering protein transduction domains themselves into a cell (Fischer et al., 2004, A stepwise dissection of the intracellular fate of cationic cell penetrating peptides. J. Biol. Chem. 279, 12625-12635), delivering DNA into a cell (Ciftci & Levy, 2001, Enhanced plasmid DNA transfection with lysosomotropic agents in cultured fibroblasts. Int. J. Pharm. 218, 81-92), and delivering proteins into a cell (Caron et al., 2004, Endosome disruption enhances the functional nuclear delivery of Tat-fusion proteinsl. Biochem. Biophy. Res. Commun. 319, 12-20). Accordingly, they could not solve problems resulted from the formation of endocytic vesicles including particles by endocytosis.

Especially, to chase particles introduced into a cell or to identify cellular structures and metabolisms, particles should be effectively exposed from endocytic vesicles to cytoplasm in a condition that physiological, biochemical, or biological environment of a cell is maintained as intact. However, the recognition about the aforesaid issues has not been noted in the academic field so far and researches related to the issues are also inactive.

Accordingly, there is a need in the art for providing technologies of chasing particles introduced into a cell or identifying cellular structures and metabolisms effectively by exposing particles effectively from endocytic vesicles to cytoplasm in a condition that physiological, biochemical, or biological environments of a cell is maintained as intact.

The inventor of the present invention has developed a cytoplasm exposure additive and a method for exposing particles delivered into a cell to cytoplasm from endocytic vesicles effectively in an intact cell where physiological, biochemical, or biological environment is maintained as undamaged.

### SUMMARY OF INVENTION

The object of the present invention is to solve aforesaid problems of the conventional technologies by providing a cytoplasm exposure additive for exposing particles delivered into a cell to cytoplasm from endocytic vesicles effectively in an intact cell where physiological, biochemical, or biological environment is maintained as undamaged.

In addition, the object of the present invention is to provide a method for exposing particles delivered into a cell to cytoplasm from endocytic vesicles effectively by allowing the cell to contact a cytoplasm exposure additive, in an intact cell where physiological, biochemical, or biological environment is maintained as undamaged.

### DETAILED DESCRIPTION OF INVENTION

To solve the problem of the formation of endocytic vesicles including particles by endocytosis, a cytoplasm exposure additive of the present invention should expose the particles from endocytic vesicles to cytoplasm effectively in an intact cell where physiological, biochemical, or biological environment is maintained as undamaged. In other words, even if the cytoplasm exposure additive exposes particles delivered into a cell to cytoplasm from endocytic vesicles, it should not affect the critical modification in physiological, biochemical, or biological activities of cellular cytoskeleton, cell membrane, subcellular organelles, or cellular materials.

In one embodiment of the present invention, the cytoplasm exposure additive comprises at least one selected from a group consisting of NDGA (Nordihydroguaiaretic acid), NEM (N-ethylmaleimide), NH₄Cl, formaldehyde, paraformaldehyde, methanol and ethanol.

Preferably, the cytoplasm exposure additive comprises at least one selected from a group consisting of NDGA, NEM, and NH₄Cl.

The present invention provides a method for exposing particles delivered into a cell to cytoplasm from endocytic vesicles comprising: introducing particles into a live cell; allowing the cell to contact a cytoplasm exposure additive which can expose the particles from endocytic vesicles to cytoplasm in the cell with maintaining its physiological, biochemical, or biological environment as undamaged; and allowing the particles to be exposed from the endocytic vesicles to the cytoplasm.

In one embodiment of the method of the present invention, the cytoplasm exposure additive comprises at least one compound selected from a group consisting of NDGA (Nordihydroguaiaretic acid), NEM (N-ethylmaleimide), NH₄Cl, paraformaldehyde, methanol and ethanol.

Preferably, the cytoplasm exposure additive comprises at least one selected from a group consisting of NDGA, NEM, and NH₄Cl.

In one embodiment of the method of the present invention, the particles may include materials having a form of particle or becoming a form of particle inside a cell. For example, the particles can be artificially synthesized and have a diameter of nanometers.

In one embodiment of the method of the present invention, the particles have a diameter between about 1 and 1,500 nm. Preferably, the particles have a diameter between about 20 and 350 nm.

### ADVANTAGEOUS EFFECTS

The present invention is advantageous in that particles delivered into cells can be effectively exposed to cytoplasm from endocytic vesicles in intact cells which maintain their physiological, biochemical, or biological environment as undamaged.

### BRIEF DESCRIPTION OF DRAWINGS

The above and other objects, features and other advantages of the present invention will be more clearly understood to those skilled in this arts from the following detailed description taken in conjunction with the accompanying drawings.

FIG. 1(A) represents pictures of the intracellular location of particles introduced into a live HeLa cell expressing ADRB2-EGFP.

FIG. 1(B) illustrates and compares a transmitted light image with original color before Prussian blue staining and a transmitted light image after fixing and Prussian blue staining of HeLa cell having introduced particles.

FIG. 2 represents pictures showing that particles in endocytic vesicles are exposed by a cytoplasm exposure additive for exposing to cytoplasm and the particles modified by dasatinib are then labeled with fluorescence by EGFP-ABL1 protein, resulting in the co-localization of the particles and the fluorescence.

### EXAMPLES

Practical and presently preferred embodiments of the present invention are illustrated more clearly as shown in the following examples. However, it should be appreciated that those skilled in the art, on consideration of this disclosure, may make modifications and improvements within the spirit and scope of the present invention. References cited in the specification are incorporated into the present invention.

### Example 1

### Identifying Whether Particles Introduced into a Cell are Located in a Vesicle

To identify whether particles introduced into a cell are located in a vesicle, an experiment was performed as follows:

To begin with, surface-modified particles were prepared. The particles used in this example can be anything that has been widely used for research and medical purpose in the art. For example, they include gold particles, beads, and etc. In this embodiment, streptavidin-conjugated superparamagnetic particles (streptavidin-magnetic particles) were produced on its own by a method known in the art (U.S. Pat. No. 5,665,582; U.S. Patent Publication No. 2003/0092029A1) or purchased from a company responsible for manufacture and sales.

Next, a labeling material was prepared to identify whether the particles introduced into a cell had been located in a vesicle. After purchasing ADRB2 (GenBank Acc. No. BC073856) gene cloned into pDonr221 from Open Biosystems, a vector expressing EGFP-bound protein was produced by a known method (Hartley, et al. (2000) DNA cloning using in vitro site-specific recombination. Genome Res. 10, 1788-1795). The designed expression vector was analyzed by sequencing. ADRB2 protein is one of GPCRs which are membrane proteins and it is known to be located in cell membrane, endosome, and lysosome, etc. Therefore, ADRB2-EGFP can be used as a fluorescently labeling material to identify whether the particles introduced into a cell are located in a vesicle.

HeLa cell (obtained from ATCC) was subcultured to 5,000˜10,000 cells/well in a 96-well plate and then transfected by the ADRB2-EGFP expression vector DNA. DNA transfection was performed by a known method, for example, lipofectamine (purchased from Invitrogen) or Fugene 6 (purchased from Roche). Next, the prepared particles were introduced into the cell transfected with DNA by a method known in the art (Josepthson, et al. (1999) High-efficiency intracellular magnetic labeling with novel superparamagnetic-Tat peptide conjugate. Bioconjug. Chem. 10, 186; Derfus, et al. (2004) Intracellular delivery of quantum dots for live cell labeling and organelle tracking Adv. Mater. 16, 961; Frank, et al. (2003) Clinically applicable labeling of mammalian and stem cells by combining superparamagnetic iron oxides and transfection agents. Radiology 228, 480; U.S. Patent Publication No. 2005/0130167; U.S. Patent Publication No. 2005/0271732).

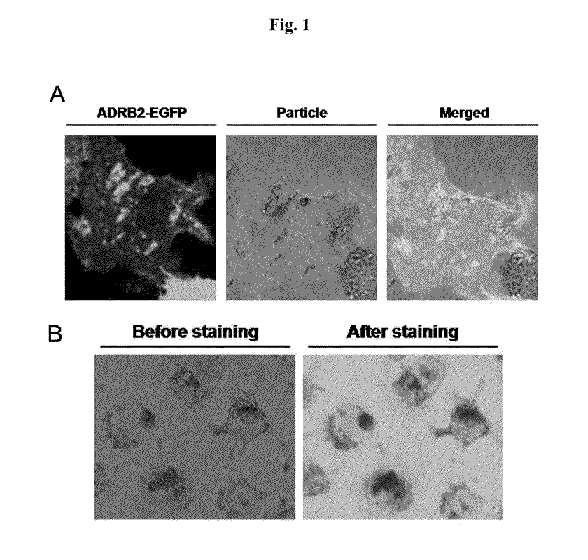

After then, it was identified whether the particles introduced into the cell and the fluorescence of ADRB2-EGFP protein were co-localized, and the intracellular location of particles introduced into the cell was then identified (FIG. 1A). The particles in the cell were observed as dark spots (black dots) under a transmission light microscope (FIG. 1A, middle). The fluorescence of ADRB2-EGFP protein was observed in a vesicle such as cell membrane, endosome, or lysosome (FIG. 1A, left). Furthermore, the co-localization between the particles introduced into the cell and the fluorescence of ADRB2-EGFP protein was identified in a vesicle (FIG. 1A, right).

Meanwhile, Prussian blue staining was performed to identify that the black dots observed in the cell were the particles introduced into the cell. In general, Prussian blue dye is used to specifically stain oxidized iron particles introduced into a cell and to observe the oxidized iron particles (Frank, J. A., Miller, B. R., Arbab, A. S., Zywicke, H. A., Jordan, E. K., Lewis, B. K., Bryant, L. H., & Bulte, J. W. M. (2003) Clinically applicable labeling of mammalian and stem cells by combining superparamagnetic iron oxides and transfection agents. Radiology 228: 480-487).

After fixing the cell having the particles by formaldehyde, the cell was observed by an optical microscope before and after Prussian blue staining in the same field. Prussian blue staining was performed by Prussian Blue Iron Stain Kit (Polysciences, Cat. No. 24199). From the Prussian blue staining, it was confirmed that the black dots observed in the cell were resulted from the oxidized iron particles (FIG. 1B). This result suggests that the particles introduced into the cell existed in the live cell, with being surrounded by an endocytic vesicle such as endosome and lysosome since the ADRB2 protein is usually located in cell membrane, endosome, or lysosome, etc.

### Example 2

### The Exposure of Particles to Cytoplasm by Cytoplasm Exposure Additives

To identify whether the cytoplasm exposure additive of the present invention can expose particles delivered into a cell to cytoplasm from endocytic vesicles, an experiment was conducted as follows:

Whether the particles delivered into a cell are exposed to cytoplasm from endocytic vesicles can be identified by various methods. For example, it can be identified by the co-localization between the fluorescence and the particles showing black dots, by using fluorescently labeling material labeling an endocytic vesicle as mentioned in Example 1.

After allowing the cytoplasm exposure additive of the present invention to contact a cell, unlike FIG. 1A, if the black dots of the particles introduced into a cell are observed in other areas except the endocytic vesicle showing the fluorescence of ADRB2-EGFP protein, the particles are considered as exposed to cytoplasm.

In addition, there is more effective method to identify the exposure to cytoplasm. In this example, it can be observed whether surface-modified particles are labeled with fluorescent materials through a mediator and whether the particles are exposed to cytoplasm. A mediator may include a single linker or a plurality of linkers available and utilized in the art. In the following example, an experiment was performed using a mediator consisting of two linkers.

Dasatinib used to treat chronic myeloid leukemia was selected for one linker constituting a mediator [Lombardo, L. J., Lee, F. Y., Chen, P., et al. Discovery of N-(2-chloro-6-methyl-phenyl)-2-(6-(4-(2-hydroxyethyl)-piperazin-1-yl)-2-methylpyrimidin-4-ylamino)thiazole-5-carboxamide (BMS-354825), a dual Src/Abl kinase inhibitor with potent antitumor activity in preclinical assays. J. Med. Chem. 47, 6658-6661 (2004); Shah, N. P., Tran, C., Lee, F. Y. Chen, P., Norris, D., Sawyers, C. L. Oerriding imatinib resistance with a novel ABL kinase inhibitor. Science 305, 399-401 (2004)]. In addition, ABL1 (GenBank Acc. No. NM_{—}198291) which binds to dasatinib was used for another linker constituting a mediator. Further, to modify the surface of particles with dasatinib, dasatinib-biotin was synthesized by a known method in the art.

For example, dasatinib-biotin was synthesized as follows: (1) After dasatinib was dissolved in a mixture of THF and DMF and then triethylamine was added, the dasatinib solution was cooled; (2) Methanesulfonyl chloride was slowly and dropwise added to the dasatinib solution and stirred overnight at room temperature; (3) After adding NaN₃, the reaction solution was stirred overnight at 50° C.; (4) The reaction solution was subjected to vacuum evaporation and then its residue was purified by column chromatography; (5) The purified product was dissolved in THF and after adding an excess of triphenylphosphine, it was stirred for 5 hours at room temperature; (6) After adding water to the reaction solution, the solution was stirred overnight at 70° C. and then subjected to vacuum evaporation; (7) The residue was dissolved in DMF under atmosphere of nitrogen, and then triethylamine was added to the solution; (8) Compound 2 dissolved in DMF was added to the solution, and the solution was stirred for 3 days at room temperature; (9) The reaction solution was subjected to vacuum evaporation and then purified by MeOH/MC column chromatography; and (10) The synthesized compound was identified by NMR or LC-MS.

EGFP-ABL1 expression vector was produced by a known method (Sambrook & Russell, 2001, Molecular Cloning, Cold Spring Harbor Laboratory Press). After HeLa cells were subcultured to 6,000 cells/well on a 96-well plate, the expression vector DNA was transfected into a cell as mentioned in Example 1.

The particles modified with dasatinib were introduced into the cell transfected with DNA in the following. The cultured HeLa cells were treated with the modified particles according to the following processes:

1) Streptavidin-magnetic particles were mixed with dasatinib-biotin and the mixture was allowed to be reacted with each other. As a result, the particles modified with dasatinib were prepared;

2) The mixture was purified by a well-known separating method in the art, for example, HGMS (High gradient magnetic separation) technology (U.S. Pat. No. 4,247,398; Melville, et. al. Direct magnetic separation of red cells from whole blood. Nature (1975) 255, 706); and

3) The prepared dasatinib-modified particles were then introduced into the HeLa cells transfected with DNA by a well-known method in the art (Josepthson, et al. (1999) High-efficiency intracellular magnetic labeling with novel superparamagnetic-Tat peptide conjugate. Bioconjug. Chem. 10, 186; Derfus, et al. (2004) Intracellular delivery of quantum dots for live cell labeling and organelle tracking Adv. Mater. 16, 961; Frank, et al. (2003) Clinically applicable labeling of mammalian and stem cells by combining superparamagnetic iron oxides and transfection agents. Radiology 228, 480; U.S. Patent Publication No. 2005/0130167; U.S. Patent Publication No. 2005/0271732).

After the cells transfected with EGFP-ABL1 expression vector DNA and having the particles modified with dasatinib were treated with NDGA (Nordihydroguaiaretic acid, Sigma), NEM (N-ethylmaleimide, Sigma), or NH₄Cl(Sigma) to the final concentration of 25˜50 μM, 5˜20 μM, or 10 mM, respectively, the live cells were observed.

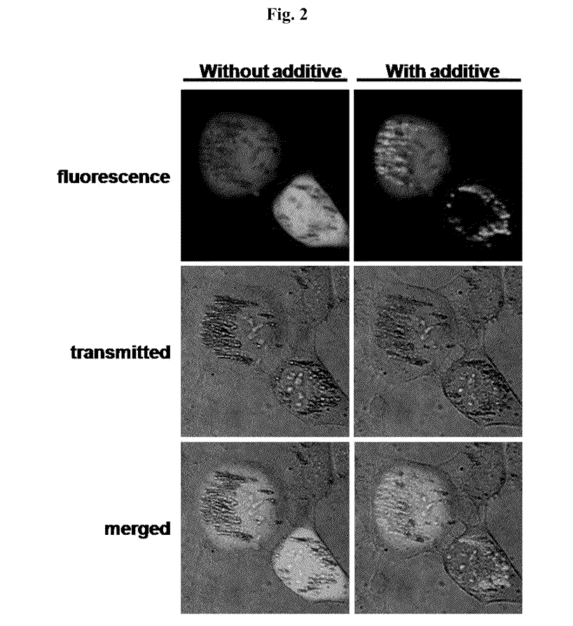

In this embodiment, Olympus fluorescence microscope, FV1000, equipped with the object lens of Uplan Apo 40×/0.85 was used to obtain transmitted images and fluorescent images in the cells. As shown in FIG. 2, since the introduced dasatinib-particles existed in an endocytic vesicle without a cytoplasm exposure additive, they could not be fluorescently labeled by EGFP-ABL1 protein and thus the black dots of the particles were not co-localized with the fluorescence of EGFP-ABL1 protein (FIG. 2, left). In contrast, if the particles in the endocytic vesicle were exposed to cytoplasm in the presence of the cytoplasm exposure additive, it was confirmed that the dasatinib-particles were fluorescently labeled by EGFP-ABL1 protein and thus the co-localization between the black dots of the particles and the fluorescence of EGFP-ABL1 protein was observed (FIG. 2, right).

In addition, the aforesaid experiment was performed using formaldehyde, paraformaldehyde, methanol or ethanol and their function as a cytoplasm exposure additive was identified. When cells are treated and allowed to be contacted with formaldehyde, paraformaldehyde, methanol or ethanol, cells cannot be observed in a living state since formaldehyde, paraformaldehyde, methanol or ethanol fixes cells. However, at least, it was confirmed that they exposed the particles to cytoplasm from endocytic vesicles effectively in intact cells which maintain their physiological, biochemical, or biological environment as undamaged.

Although the present invention has been illustrated and described with reference to the exemplified embodiments of the present invention, it should be understood that various changes, modifications and additions to the present invention can be made without departing from the spirit and scope of the present invention.

1. A cytoplasm exposure additive for exposing particles delivered into a cell to cytoplasm from endocytic vesicles comprising: at least one selected from a group consisting of NDGA (Nordihydroguaiaretic acid), NEM (N-ethylmaleimide), NH₄Cl, formaldehyde, paraformaldehyde, methanol and ethanol.

2. The cytoplasm exposure additive as claimed in claim 1, wherein comprising at least one selected from a group consisting of NDGA, NEM, and NH₄Cl.

3. A method for exposing particles delivered into a cell to cytoplasm from endocytic vesicles comprising: introducing particles into a live cell; allowing the cell to contact a cytoplasm exposure additive which can expose the particles from endocytic vesicles to cytoplasm in the cell with maintaining its physiological, biochemical, or biological environment as undamaged; and allowing the particles to be exposed from the endocytic vesicles to the cytoplasm.

4. The method as claimed in claim 3, wherein the cytoplasm exposure additive comprises at least one selected from a group consisting of NDGA (Nordihydroguaiaretic acid), NEM (N-ethylmaleimide), NH₄Cl, formaldehyde, paraformaldehyde, methanol and ethanol.

5. The method as claimed in claim 3, wherein the cytoplasm exposure additive comprises at least one selected from a group consisting of NDGA, NEM, and NH₄Cl.

6. The method as claimed in claim 3, wherein the particles include materials having a form of particle or becoming a form of particle inside a cell.

7. The method as claimed in claim 3, wherein the particles have a diameter between about 1 and 1,500 nm.

8. The method as claimed in claim 7, wherein the particles have a diameter between about 20 and 350 nm.
